(19) United States
(12) Patent Application Publication
(10) Pub. No.: US 2024/0038355 A1
(43) Pub. Date: Feb. 1, 2024

(54) METHOD FOR PROVIDING A CLIENT WITH AN INFORMATION REGARDING MAIN CHARACTERISTICS OF A PHARMACEUTICAL PRODUCT AND/OR AN ANALOGUE THEREOF FOLLOWED BY DISPENSING THEREOF IN AN AUTOMATED PHARMACY STORE USING AT LEAST ONE HARDWARE PROCESSOR AND A MEMORY FOR STORING COMPUTER-EXECUTABLE INSTRUCTIONS OR A MOBILE APPLICATION, AND A SYSTEM FOR PERFORMING SAID METHOD

(71) Applicants: Serhii Yevhenovych Monashov, Kyiv (UA); Myroslav Illych Yakibchuk, Kyiv (UA); Fred M Mosely, Solon, OH (US); Mykhailo Viktorovych Panov, Volnovaha (UA)

(72) Inventors: Serhii Yevhenovych Monashov, Kyiv (UA); Myroslav Illych Yakibchuk, Kyiv (UA); Fred M Mosely, Solon, OH (US); Mykhailo Viktorovych Panov, Volnovaha (UA)

(21) Appl. No.: 17/931,763

(22) Filed: Sep. 13, 2022

(30) Foreign Application Priority Data
Jul. 28, 2022 (UA) A 2022 02708

(51) Int. Cl. G16H 20/13; G16H 70/40; G06T 13/40; G10L 15/22; G06F 3/16

(52) U.S. Cl. CPC G16H 20/13; G16H 70/40; G06T 13/40; G10L 15/22; G06F 3/167; G06Q 20/18

(57) ABSTRACT

A group of inventions relates to a field of information technologies, namely, to a method for providing a client with an information regarding main characteristics of a pharmacy product and/or an analogue thereof in an automated pharmacy store or in a mobile application followed by dispensing the pharmacy product and/or the analogue thereof in the automated pharmacy store, and to a system for performing said method, which enable to achieve a technical effect that lies in a maximum quick, accurate and reliable processing of the client's incoming request, and, thus, a timely providing a maximum correct information regarding the main characteristics of the pharmacy product and/or the analogue thereof by means of a virtual assistant having pre-recorded programs of algorithms and fully uploaded pharmacology databases which are capable of being updated on-line, that does not require additional settings from the client and may operate in an autonomous mode, as well as in providing a high trust level of the virtual assistant from the client's side due to its maximum visual similarity to a real human and that is capable of providing the information to the client both verbally and visually at the same time.

### CROSS-REFERENCE TO RELATED APPLICATION

This application claims priority to Ukrainian Application No. a 2022 02708, filed Jul. 28, 2022, which is hereby incorporated by reference in its entirety.

### FIELD OF THE INVENTION

A group of inventions relates to a field of information technologies, namely, to a method for providing a client with an information regarding main characteristics of a pharmacy product and/or an analogue thereof in an automated pharmacy store or in a mobile application followed by dispensing the pharmacy product and/or the analogue thereof in the automated pharmacy store, and a system for performing said method.

### PRIOR ART

A high-quality and quick provision of pharmaceutical services is one of main prerogatives of a modern health care system.

In spite of a wide development of pharmaceutical drug retail networks, a pharmacy industry nowadays is rather often characterized by queues, where a client has to spend a long time which is related to the fact that according to a legislation, almost in all countries worldwide, pharmacy stores are not allowed to operate without a physical presence of a pharmacist or a provisor who are able to provide a required information and recommendations regarding main characteristics of pharmaceutical drugs, to suggest analogues, and to provide any other consultation services as per the client's request.

In recent years, in order to optimize and to shorten a duration for processing the client's request, automated pharmacy stores become increasingly popular, which mainly function to store pharmacy products such as packages, blisters, packets, vials etc. at defined temperature and humidity ranges; to accept the products for storage; to provide a user interface for interaction with the pharmacist or provisor, thereby providing a convenient search of the products according to their name, pharmacological groups, code, bar code etc.; to dispense the selected products quickly. Existence of such automated pharmacy stores accelerates the clients ‘servicing process significantly, however, a necessity of on-line consultations by a physical pharmacist person introduces essential limitations which are related to the fact that the automated pharmacy stores are not able to operate 24/7, to a risk of termination of the consultation if there is no Internet connection, to the fact that a quality of the provided consultation services depends on the pharmacist's level of professional skills etc. In order to overcome said drawbacks, various chat-bots became increasingly used to consult the clients within networks of the automated pharmacy stores, the chat-bots are programs which are capable of imitating a real conversation with the client by means of text or audio messages and which use artificial intelligence elements or operate based on programmed multiple selection scenarios.

The prior art teaches automated pharmacy stores which utilize various methods for consulting the clients such as conducting on-line consultations by a physical pharmacist or provisor person, use of chat-bots using artificial intelligence elements or chat-bots which operate based on programmed scenarios etc., and the inventors have chosen several technical solutions among them which are the closest ones to the proposed group of inventions in terms of a set of essential features.

So, a U.S. Pat. No. 8,849,449 B2 dated Sep. 30, 2014 teaches an automated pharmacy store, a method and a system which are intended to dispense drugs in a quick, convenient and safe manner. According to the proposed technical solution, a prescription with drugs prescribed to a user is generated, the prescription comprising a human or a computer readable description of the drugs and a patient data. The prescription is provided to a robotic dispenser operable to recognize the human or the computer readable description followed by validating and dispensing the corresponding drugs. Therewith, the automated pharmacy store comprises a client interface, a teleconferencing or video conferencing means which provide a communication between the patient and a physical person being a prescription validation agent, and a prescription scanning means. Preferably, a licensed pharmacist acts as the physical person being the prescription validation agent which is able to control the dispenser and to approve each prescription if necessary. In embodiments of the invention which include a consultation or the pharmacist's involvement, upon approval of all the patient information, the latter is communicated with a call-center pharmacist by means of the video conference. Furthermore, the patient is enabled to communicate with the call-center pharmacist on their own at any moment by pressing a button “Call the pharmacist” which is arranged on the client interface of the dispenser. The video conference is usually conducted by means of standard video conference systems via Internet, and several video cameras are mounted within the dispenser in order to enable the pharmacist to observe both the patient and the drugs, while they are still located in the dispenser, simultaneously. In order to display the pharmacist on a screen in real-time, a standard web camera is used. A voice conference is conducted via a standard microphone and a speaker which are embedded into the dispenser, or, in order to provide the patient's confidentiality, via a standard handset connected to the dispenser. Drawbacks of conduction of the on-line consultations for the patient by the physical pharmacist person by means of the video and teleconferences lie in an increased risk of termination of such consultations due to a lack of the Internet connection or a phone connection, in the fact that an operation time of such automated pharmacy stores is limited by the pharmacist's working hours, and, thus, in their inability to operate 24/7, in that a quality of the provided consultation services depends on the pharmacist's level of professional skills. Furthermore, the technical solution implies conduction of the consultations directly during the use of the automated pharmacy stores and it does not enable to receive a preliminary information regarding an availability of the drugs in a certain automated pharmacy store by means of a mobile application which could allow the patient to avoid unnecessary visits to the pharmacy store and, thus, to save time considerably.

A U.S. Pat. No. 7,630,788 B1 dated Dec. 8, 2009 teaches a pharmaceutical system in which a client is provided with a pharmaceutical care by a remote professional serving multiple pharmacies. The technical solution provides a device comprising at least one prescription input device adapted to receive a prescription data corresponding to a prescription that is presented by the client, at least one robotic device for handling medical drugs, at least one pharmacy computer in an operative connection with the at least one prescription input device and the at least one robotic device for handling medical drugs, wherein the at least one pharmacy computer is operative to communicate the prescription data to a remote pharmacist terminal which, by means of the pharmacy computer, controls the at least one robotic device for handling the medical drug to prepare a medical drug corresponding to the prescribed prescription responsive to a communication with the pharmacist terminal. The device further comprises at least one client communication device that is operatively connected to the at least one pharmacy computer that provides a final communication between the pharmacist and the client that presented the prescription at a location, and that represents an audio communication or an audiovisual communication device. A drawback of the proposed technical solution, as in the previous case, lies in that it is necessary to conduct audio- or audiovisual consultations with the client by the remote pharmacist, thereby limiting the operation duration of the pharmaceutical system taught by the patent strictly by working hours of the remote pharmacist, while a presence and a quality of the communication therewith depend on the operation stability and speed of the network, as well as the fact that the quality of the provided pharmaceutical services depends on the pharmacist's professional skills who provides these services.

An application CN110992597 A dated Apr. 10, 2020 teaches a device and a method for selling drugs using an artificial intelligence based on Internet technologies, according to which, a client selects one of three drug purchasing modes via a main interface of a touch screen of the device and executes operations according to the selected mode, wherein the drug purchasing modes are as follows: logging in a self-servicing system, a purchasing instruction, logging in a consultation on the main interface according to own needs. According to the proposed technical solution, the client logs in a self-servicing interface for drug packing via a self-servicing module for drug packing, identifies a drug packaging code by means of a drug packaging code identification module, and receives a list of purchased drugs followed by approving the list of drugs via a purchased drug approval module and transmitting an information about the purchased drug to a processor via a purchased drug results formation module. Therewith, in one of embodiments of the invention, the client enters the pharmacist consultation interface via a drug purchase instruction module, registers via a registration module, and obtains an on-line consultation using a video communication with the pharmacist via the consultation module. A drawback of the proposed technical solution lies in that the drug purchasing process is based on the self-servicing principle and does not comprise providing the client with the pharmacist's consultations at all steps of the drug purchase, besides, the consultations are conducted by the pharmacist that works remotely exclusively as per a client's separate request after registration in the corresponding module of the device which is accompanied by significant time costs during execution of this operation, as well as by instability of communication with the pharmacist, since this communication may be terminated at any moment if there is no Internet connection.

A system and a method for providing a client with a medical health care information by means of a virtual assistant according to a U.S. Ser. No. 11/029,918 B2 dated Jun. 8, 2021 are taken as the closest analogue of the group of inventions. According to the proposed technical solution, the system comprises at least one hardware processor and a memory for storing computer executable instructions which cause performing the following operations: causing a display of a dialog client interface along with a visual display of the virtual assistant that is set by the client, receiving a client's input data based on a voice message and/or a keyboard input and/or a touch screen data input, presenting a first response in the dialog client interface in a form of a first message from the virtual assistant, displaying an assumption received from a context of the client's input data in the dialog client interface, wherein the assumptions are displayed separately from the first message, receiving a modification of the displayed assumption and, in response to the modification, determining a second response to the client's input data at least partially based on client's medical records and on the modified assumption, updating the dialog client interface in order to replace the first message with a second message from the virtual assistant that represents the second response. Therewith, the virtual assistant may comprise an avatar that may be visually similar to a human face or may be an animated character which can take any shapes and images and remind of a human. A drawback of the proposed technical solution lies in that it is necessary for the client to set the virtual assistant which requires additional time costs and may be a source of human-related errors. Besides, the proposed technical solution is characterized by an insufficient accuracy of processing the client's incoming requests which require future modifications of the assumptions which are made by the virtual assistant, thereby leading to a significant increase of the duration of processing the request, wherein the response to the request is provided to the client in a form of a text message only and does not imply a verbal response of the system by means of the virtual assistant, thereby negatively affecting a servicing level and the client's satisfaction in general, as well as it may cause information perception difficulties for clients suffering from vision disorders. Furthermore, the virtual assistant made in the form of the avatar that reminds of the human face and that provides the response to the request in the form of the text message only is not perceived by the client as a full substitution of a real pharmacist, thereby negatively affecting the trust level of the virtual assistant.

### SUMMARY OF THE INVENTION

The claimed group of inventions is based on a task to develop a method and a system which are reliable and 24/7 available due to a lack of a human-related factor influence, algorithmically simple and clear, the method and the system are for providing a client with an information regarding main characteristics of a pharmacy product and/or an analogue thereof in an automated pharmacy store or in a mobile application using at least one hardware processor and a memory for storing computer executable instructions followed by dispensing the pharmacy product and/or the analogue thereof in the automated pharmacy store, which enable to achieve a technical effect that lies in a maximum quick, accurate and reliable processing of a client's incoming request, and, thus, in a timely providing a maximum correct information regarding the main characteristics of the pharmacy product and/or the analogue thereof by means of a virtual assistant having pre-recorded programs of algorithms and fully uploaded pharmacology databases which are capable of being updated on-line, that does not require additional settings from the client and may operate in an autonomous mode, as well as in providing a high trust level of the virtual assistant from the client's side due to its maximum visual similarity to a real human and that is capable of providing the information to the client both verbally and visually at the same time.

The posed task is resolved by the fact that the method for providing the client with the information regarding the main characteristics of the pharmacy product and/or the analogue thereof in the automated pharmacy store or in the mobile application using at least one hardware processor and the memory for storing the computer executable instructions followed by dispensing the pharmacy product and/or the analogue thereof in the automated pharmacy store comprises:
causing, by the client, a display of a dialog client interface via a graphical user interface (GUI) module simultaneously with a visual three-dimensional image on a screen of the virtual assistant having recorded programs of algorithms and uploaded databases which are capable of being updated on-line, wherein the update rate of the three-dimensional image of the virtual assistant is at least 60 Hz/sec, thereby providing its maximum similarity to the real human, i.e., the virtual assistant is a fully visualized human prototype which makes the interaction between the client and the virtual assistant much easier and helps to increase the trust level of the latter, and the recorded programs of algorithms and the uploaded databases which preferably are pharmacology databases and do not require additional settings from the client, enable to provide the client with the information regarding the main characteristics of the pharmacy product and/or the analogue thereof in the autonomous mode in a maximum quick and accurate manner even if there is no Internet connection. Therewith, the recorded programs of algorithms of the virtual assistant, which are implemented by means of, e.g., an embedded application software Firmware for automated pharmacy stores and a mobile application App for personal devices such as smartphones or laptops, provide the maximum accuracy of processing of the client's requests and minimize a percentage of their incorrect perception. According to the researches conducted by the inventors, it has been established that the percent of the incorrect processing of the client's requests that led to provision of the incorrect information regarding the main characteristics of the pharmacy product and/or the analogue thereof that has not corresponded to the client's incoming request partially or completely was not more than 5%, i.e., the accuracy of provision of the information by the virtual assistant by means of the recorded programs of algorithms is at least 95% that corresponds to the accuracy of provision of the information by a real pharmacist. Also, the inventors have conducted research to compare a satisfaction and trust level of the clients in case of processing of their requests by the virtual assistant that represented a completely visualized human prototype capable of providing responses both verbally and textually on the screen simultaneously, and the virtual assistant that represented a human avatar in a two-dimensional image that provided responses merely textually and without any verbal support. The researches have shown that the satisfaction and trust level, when the clients were receiving the information regarding the main characteristics of the pharmacy product and/or the analogue thereof using the three-dimensional image of the virtual assistant that represented the completely visualized human prototype capable of providing responses verbally, thereby engaging in a dialog with the client, was higher in 85% of cases as compared to the satisfaction level when receiving the information merely textually and in case of the two-dimensional representation of the virtual assistant, and 10% of respondents have not given importance to the form of the information provision and 5% of respondents have given importance to the second option of the information provision, i.e., by means of the two-dimensional avatar and text;
receiving the incoming request from the client by verbal input thereof by means of a microphone. The client's incoming request relates to the information regarding the main characteristics of the pharmacy product, namely, to a mode of action of the medical drug, an active substance which it is based on, side effects caused by use of the medical drug, age restrictions as to use of the drug, contraindications, an expiration date of the drug, a country of manufacture, an existence of analogues etc. Therewith, the client's incoming verbal request is compiled in a form of completed sentences, and in order to increase the accuracy of its subsequent processing, the verbal input of the request also may be accompanied by a simultaneous input of the textual request on the touch screen and corrected during the dialog with the virtual assistant via the touch screen or a scanner. It should be understood that the examples of possible options for receiving the incoming request are merely illustrative and do not limit a scope of rights provided by the invention;
determining parameters of the client's incoming request by means of a direct speech recognition module by processing keywords of the request using the recorded programs of algorithms of the virtual assistant;
receiving a data from the uploaded databases which are configured to exchange a data with external databases at a data transfer rate of at least 4 MBit/sec. The uploaded databases comprise databases of pharmacy networks that comprise data regarding an availability and a variety of pharmacy products in the pharmacy store, databases of electronic prescriptions of medical institutions which, together with the data from the databases of the pharmacy networks, enable to verify a validity of the prescription made by a physician and an information as to whether this prescription is used repeatedly for obtaining drugs, and pharmacological knowledge databases which enable providing the client with a maximum full information regarding the main characteristics of the pharmacy product and/or the analogue thereof, wherein the possibility of the quick update of said databases by exchange the data with the external databases at the data exchange rate of at least 4 MBit/sec enables to increase a rate of providing the information in response to the incoming requests from the clients significantly;
processing a client's personal data by means of a personal data processing module, the processing comprises processing a client's ID card data or a passport and a prescription prescribed by the physician by scanning them by means of the scanner provided in the automated pharmacy store or photographing by means of a camera that is provided in a mobile phone, a laptop or another alternative personal device in case the mobile application is used. The personal data processing module further comprises a Face ID system that, by means of an embedded video camera, enables to recognize a face of the client and, thus, to identify his/her identity by comparing an image taken by the video camera with a passport or ID card photo and, therefore, makes it impossible to use the passport or the ID card by unauthorized persons. The received passport data such as, e.g., age and sex of the client, enables to define that it is necessary to use age restrictions, e.g., for persons over sixty five years old or under eighteen years old, when selecting a certain pharmacy product, in particular, a medical drug, or when choosing the analogue, and to reveal if there are any possible contraindications as to use thereof, as well as to provide the client exactly with the information that he/she requires in a maximum accurate fashion upon a preliminary receipt of the data from the uploaded databases. Besides, a central server stores a history of all previous client's incoming requests and orders, as well as their periodicity, which become available upon scanning of the client's ID card or passport and which serve as a basis for the virtual assistant to determine, by means of the recorded programs of algorithms, with at least 99% accuracy, that a certain medical drug is over from a moment of its last purchase in the automated pharmacy store and to propose this drug or the analogue thereof to the client in a maximum quick fashion. Additionally, scanning or photographing of the prescription prescribed by the physician and receiving the corresponding data regarding the prescription from the databases of the pharmacy networks and/or from the databases of the electronic prescriptions of the medical institutions enables to verify whether the prescription is valid and whether the medical drugs according to this prescription have been received in any other pharmacy store. It should be understood that the examples of the personal data processing are merely illustrative and do not limit a scope of rights provided by the invention.

Next steps of the method comprise:
providing the client with the response to the incoming request in a form of a direct verbal response of the virtual assistant by means of a module for providing the response to the incoming request having an embedded speaker, while at the same time displaying a configuration of the client's order on the screen of the dialog client interface of the GUI module, thereby enhancing the client's level of perception of the information significantly due to the simultaneous verbal and visual display thereof;
selecting, by the client, an option for completing the order displayed on the screen of the dialog client interface of the GUI module;
paying, by the client, for the selected order by means of a payment processing module, the payment is made by means of external payment systems VISA and/or MasterCard, if the automated pharmacy store is used, and/or on-line payment systems APPLE PAY and/or GOOGLE PAY, if the payment is made in the mobile application, or in cash by means of a bill acceptor embedded into the automated pharmacy store that is the only one and backup off line payment method if there is no Internet connection;
dispensing the pharmacy product and/or the analogue thereof in the automated pharmacy store by means of a pharmacy product dispensing module, the dispensing is performed by means of a dispenser embedded into the automated pharmacy store or by means of QR code, if the mobile application is used. When placing a pre-order using the mobile application, the client receives the QR code of the processed order and, when if the automated pharmacy store is used, passes directly to dispensing of the pharmacy product, if a pre-payment has been made via the mobile application, or pays for the order by means of the payment system in the automated pharmacy store, and then receives the corresponding pharmacy product in a pharmacy product dispensing module. Use of the QR code that implies placing the pre-order by means of the mobile application enables to shorten the time spent by the client near the automated pharmacy store significantly and to use the latter exclusively as a pharmacy product dispensing point, wherein locations of all of the automated pharmacy stores are displayed in the mobile application which enables the client to select the closest dispensing point. Besides, if the dispenser is not able to dispense the pharmacy product for technical reasons, the pharmacy product dispensing module, by means of an embedded printer, will print the QR code for the client to receive his/her order in any other automated pharmacy store.

Therewith, the pharmacy product is a pharmaceutical drug and/or an analogue thereof and/or personal hygienic items and/or medical items and/or disinfectants and/or cosmetic agents.

Therewith, the external databases represent databases of the pharmacy networks and/or databases of electronic prescriptions of medical institutions and/or pharmacological knowledge databases and/or payment systems VISA and/or MasterCard and/or APPLE PAY and/or GOOGLE PAY.

Therewith, the verbal response of the three-dimensional virtual assistant is provided by a computer program that is responsible for compliance with an order of actions of the virtual assistant in real-time, thereby enabling to provide the client with the information instantly upon receipt of the corresponding incoming request.

Therewith, the client's selection of the order displayed on the screen of the dialog client interface of the GUI module is a voice selection and/or a selection made by pushing buttons on the keyboard and/or a selection made by means of the touch screen.

A second invention of the group of inventions is a system for providing a client with an information regarding main characteristics of a pharmacy product and/or an analogue thereof in an automated pharmacy store or in a mobile application, the system is configured to use at least one hardware processor and a memory for storing computer executable instructions followed by dispensing the pharmacy product and/or the analogue thereof in the automated pharmacy store, the system comprises:
a graphical user interface (GUI) for providing a visual three-dimensional image on a screen of a virtual assistant having recorded programs of algorithms and uploaded databases which are capable of being updated on-line, wherein an update rate of the three-dimensional image of the virtual assistant is at least 60 Hz/sec, thereby providing its maximum similarity to a real human, i.e., the virtual assistant is a fully visualized human prototype which makes the interaction between the client and the virtual assistant much easier and helps to increase the trust level of the latter, and the recorded programs of algorithms and the uploaded databases which preferably are pharmacology databases and do not require additional settings from the client, enable to provide the client with the information regarding the main characteristics of the pharmacy product and/or the analogue thereof in the autonomous mode in a maximum quick and accurate manner even if there is no Internet connection. Therewith, the recorded programs of algorithms of the virtual assistant, which are implemented by means of, e.g., an embedded application software Firmware for automated pharmacy stores and a mobile application App for personal devices such as smartphones or laptops, provide the maximum accuracy of processing of the client's requests and minimize a percent of their incorrect perception;
a client's direct speech recognition module having a microphone embedded therein for determining parameters of a client's incoming verbal request;
a module for uploading databases which are configured to exchange a data with external databases at a data transfer rate of at least 4 MB it/sec. The uploaded databases comprise databases of pharmacy networks, databases of electronic prescriptions of medical institutions, and pharmacological knowledge databases which enable providing the client with a maximum full information regarding the main characteristics of the pharmacy product and/or the analogue thereof, wherein the possibility of the quick update of said databases by their exchange with the external databases at the data exchange rate of at least 4 MBit/sec enables to increase a rate of providing the information in response to the incoming requests from the clients significantly;
a client's personal data processing module comprising an embedded scanner, if the automated pharmacy store is used, or a camera, if a mobile phone, a laptop or any other personal device is used, which help to perform reading and further processing of a client's ID card or passport data, as well as a drug prescription prescribed by a physician. The module further comprises a Face ID system that, by means of an embedded video camera, enables to recognize a face of the client and, thus, to identify his/her identity by comparing an image from the video camera with a passport or ID card photo and, therefore, makes it impossible to use the passport or the ID card by unauthorized persons;
a module for providing the client with a response to the incoming request in a form of a direct verbal response of the virtual assistant, the module comprises an embedded speaker;
a payment processing module comprising a reader of magnetic bank and credit cards, including chipped ones, of payment systems VISA and/or MasterCard, a bill acceptor for a cash payment in case of use of the automated pharmacy store, as well as on-line payment systems APPLE PAY and/or GOOGLE PAY in case the payment is made in the mobile application;
a pharmacy product dispensing module comprising a robotic dispenser for dispensing the product by the automated pharmacy store and a printer for printing a QR code for the client to receive his/her order in any other automated pharmacy store, if the dispenser is not able to dispense the pharmacy product for technical reasons. The pharmacy product dispensing module of the automated pharmacy store further comprises a reader of the QR code received by the client when placing a pre-order in the mobile application upon payment therefor in the mobile application or requiring the payment to be made in the payment processing module of the automated pharmacy store.

Therewith, the pharmacy product is a pharmaceutical drug and/or an analogue thereof and/or personal hygienic items and/or medical items and/or disinfectants and/or cosmetic agents.

Therewith, the external databases represent databases of the pharmacy networks and/or databases of electronic prescriptions of medical institutions and/or pharmacological knowledge databases and/or payment systems VISA and/or MasterCard and/or APPLE PAY and/or GOOGLE PAY.

### BRIEF DESCRIPTION OF THE DRAWINGS

A possibility of implementation of the group of inventions is illustrated by the drawings, which show the following:

FIG. 1 shows a general algorithm for implementation of the method for providing the client with the information regarding the main characteristics of the pharmacy product and/or the analogue thereof in the automated pharmacy store or in the mobile application using at least one hardware processor and the memory for storing the computer executable instructions followed by dispensing the pharmacy product and/or the analogue thereof in the automated pharmacy store, according to a preferable embodiment of the invention.

FIGS. 2A and 2B show a general diagram of main modules of the system for providing the client with the information regarding the main characteristics of the pharmacy product and/or the analogue thereof in the automated pharmacy store or in the mobile application, the system is configured to use at least one hardware processor and the memory for storing the computer executable instructions followed by dispensing the pharmacy product and/or the analogue thereof in the automated pharmacy store, according to a preferable embodiment of the invention.

FIGS. 3A and 3B schematically show main steps of providing the client with the information regarding the main characteristics of the pharmacy product and/or the analogue thereof in the automated pharmacy store or in the mobile application followed by dispensing of the pharmacy product and/or the analogue thereof in the automated pharmacy store, and their relations with main elements of the system which help to perform them.

FIG. 4 shows a general view of the automated pharmacy store for dispensing the pharmacy product and/or the analogue thereof.

The illustrative drawings that explain the claimed group of inventions as well as the mentioned particular exemplary embodiments are in no way intended to limit the scope of rights appended hereto but to explain the essence of the group of inventions.

### IMPLEMENTATION OF THE GROUP OF INVENTIONS

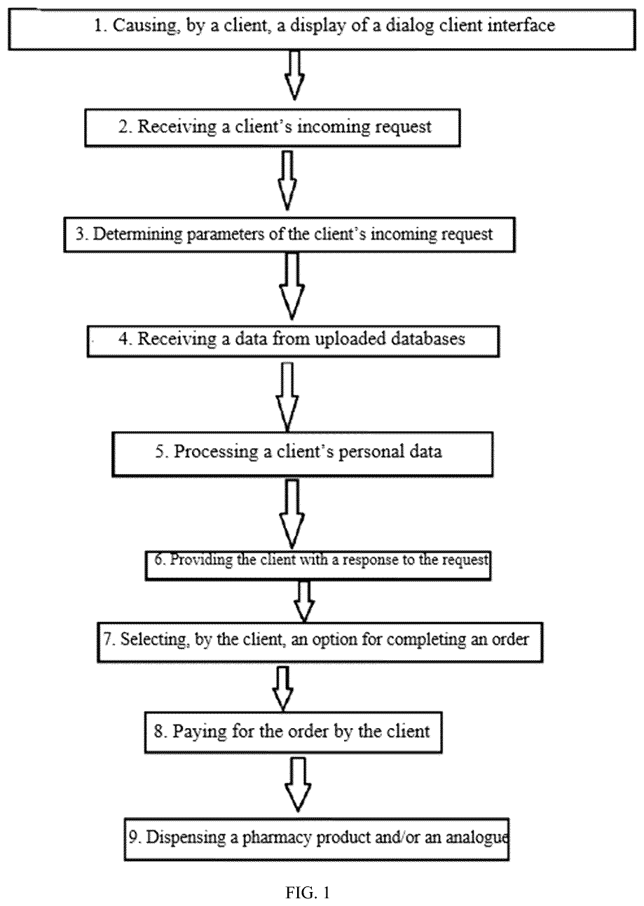

FIG. 1 illustrates a general algorithm for implementation of a method for providing a client with an information regarding main characteristics of a pharmacy product and/or an analogue thereof in an automated pharmacy store or in a mobile application using at least one hardware processor and a memory for storing computer executable instructions followed by dispensing the pharmacy product and/or the analogue thereof in the automated pharmacy store, according to a preferable embodiment of the invention. The illustrated algorithm of the method for providing information comprises the following steps. At a step 1, the client causes a display of a dialog client interface via a GUI module simultaneously with providing a visual three-dimensional image on a screen of a virtual assistant together with recorded programs of algorithms and uploaded databases which are configured to be updated on-line, wherein an update rate of the three-dimensional image of the virtual assistant is at least 60 Hz/sec, thereby providing its maximum similarity to a real human, i.e., the virtual assistant is a fully visualized human prototype which makes an interaction between the client and the virtual assistant much easier and helps to increase a trust level of the latter. At a step 2, a client's incoming request is received by means of its verbal inputting that also may be accompanied by the simultaneous inputting of a textual request on the touch screen in order to increase an accuracy of its future processing, at a step 3, the virtual assistant determines parameters of the client's incoming request by means of a direct speech recognition module by processing keywords of the request using the recorded programs of algorithms of the virtual assistant. A step 4 comprises receiving a data from the uploaded databases which represent databases of pharmacy networks, databases of electronic prescriptions of medical institutions, and pharmacology knowledge databases which provide the client with a maximum complete information regarding the main characteristics of the pharmacy product and/or the analogue thereof. Therewith, said databases are configured to be updated with the external databases at data exchange rate of at least 4 MBit/sec, thereby providing a maximum quick provision of the information to the client according to his/her incoming request. At a step 5, a client's personal data such as a client's ID card or passport data, as well as a drug prescription prescribed by a physician, is processed, thereby enabling to determine a presence of certain restrictions related to the client's age or sex, as well as to verify a validity of the prescribed prescription and whether the drug according to this prescription has been given at another pharmacy store. Furthermore, the step 5 comprises identifying the client's identity by comparing an image taken by the video camera to a photo in the passport or in the ID card, thereby making it impossible to use the passport or the ID card by unauthorized persons. A step 6 comprises providing the client with a response in a form of a direct verbal response of the virtual assistant by means of a module for providing the response to the incoming request with an embedded speaker, while at the same time displaying a configuration of a client's order on the screen of the dialog client interface of the GUI module, thereby enhancing the client's level of perception of the information significantly, since it is displayed both verbally and visually simultaneously. At a step 7, the client selects an option for placing the order that is displayed on the screen of the dialog client interface of the GUI module using a voice, pushing buttons on the keyboard or by means of the touch screen, at a step 8, the client makes a payment for the selected order by means of a payment system VISA and/or MasterCard, or cash payment by means of a bill acceptor, if the automated pharmacy store is used, or payment systems APPLE PAY and/or GOOGLE PAY, if the mobile application is used. A step 9 comprises dispensing the pharmacy product and/or the analogue thereof by means of the dispenser that is embedded into a pharmacy product dispensing module of the automated pharmacy store or by means of a QR code in case a pre-order is placed in the mobile application. The described algorithm of the method for providing the information is merely illustrative and it does not limit the scope of rights of the invention.

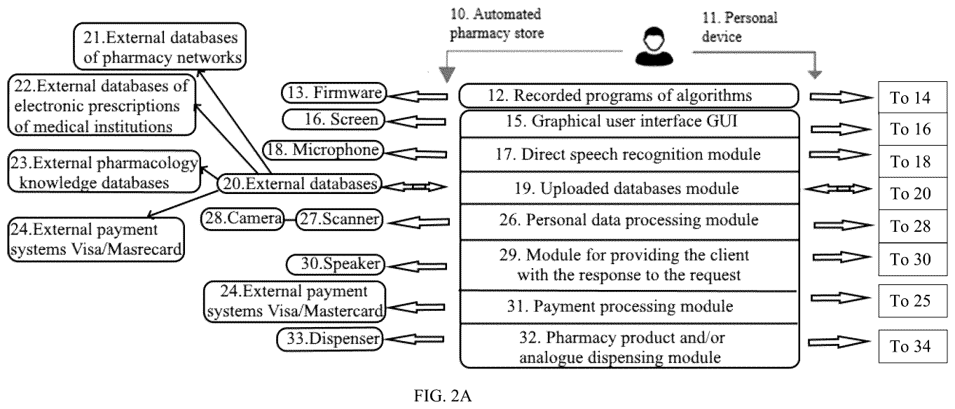

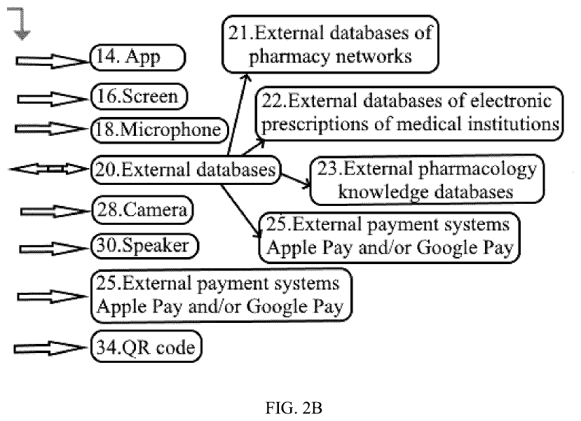

FIGS. 2A and 2B illustrate a general diagram of main modules of a system for providing a client with an information regarding main characteristics of a pharmacy product and/or an analogue thereof in an automated pharmacy store 10 or in a mobile application of personal devices 11 such as tablets, smartphones, laptops, the system is configured to use at least one hardware processor and a memory for storing computer executable instructions followed by dispensing the pharmacy product and/or the analogue thereof in the automated pharmacy store, according to a preferable embodiment of the invention. According to the illustrated diagram, the system for providing the client with the information comprises a GUI module 15 for providing a three-dimensional image on a screen 16 of a virtual assistant having programs of algorithms 12 recorded thereon, wherein an update rate of the three-dimensional image of the virtual assistant is at least Hz/sec. Therewith, the recorded programs of algorithms 12 of the virtual assistant are provided owing to an embedded application program Firmware 13 for the automated pharmacy stores 11 and to the mobile application App 14 for the personal devices 12 such as smartphones or laptops. The system further comprises a direct speech recognition module 17 with an embedded microphone 18 for determining parameters of a client's incoming verbal request, an uploaded databases module 19, the databases are configured to exchange data with external databases 20 which represent external databases of pharmacy networks 21, external databases of electronic prescriptions of medical institutions 22, external pharmacology knowledge databases 23, external payment systems Visa/MasterCard 24 for making payments in the automated pharmacy stores, and external payment systems APPLE PAY and/or GOOGLE PAY 25, if the payment is made in the mobile application. The system comprises a personal data processing module 26 having a scanner 27 and a camera 28 which is a video camera in case of the automated pharmacy store 10 and a photo and video camera in case the mobile application of the personal device 11 is used which help to read and to further process a client's ID card or passport data, as well as the drug prescription prescribed by the physician, and to identify the client's identity by comparing an image taken by the video camera to a photo in the ID card or passport, a module 29 for providing the client with a response to the request in a form of a direct verbal response of the virtual assistant with an embedded speaker 30, a payment processing module 31 for processing payments by means of the external payment system Visa/MasterCard 24, if the automated pharmacy store 10 is used, and the external payment system APPLE PAY and/or GOOGLE PAY 25, if the payment is made in the mobile application of the mobile device 12. The system further comprises a pharmacy product and/or an analogue thereof dispensing module 32 that comprises a dispenser 33 in the automated pharmacy store and a printer 34 for printing a QR code for the client to receive his/her order in any other automated pharmacy store, if the dispenser 33 is not able to dispense the pharmacy product for technical reasons in the automated pharmacy store which the client referred to.

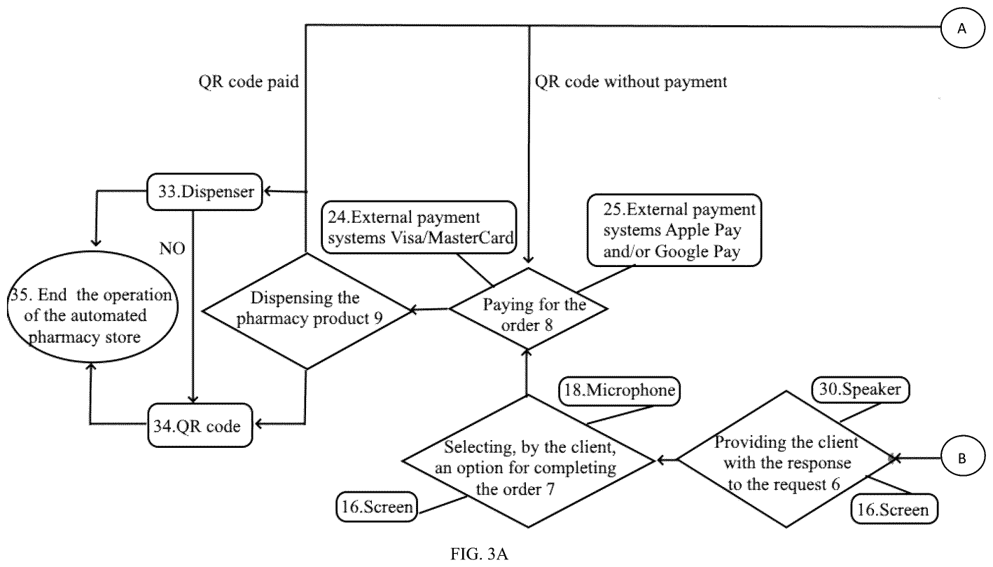

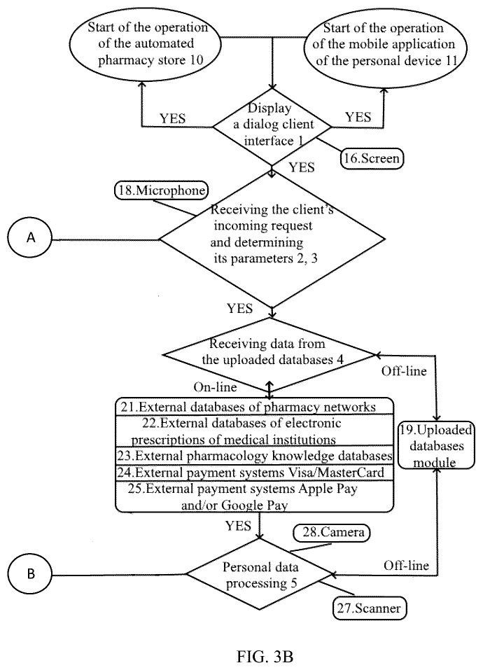

FIGS. 3A and 3B schematically show main steps of providing the client with the information regarding the main characteristics of the pharmacy product and/or the analogue thereof in the automated pharmacy store or in the mobile application followed by dispensing the pharmacy product and/or the analogue thereof in the automated pharmacy store, and their relations with main elements of the system which help to perform the same. The operation of the automated pharmacy store 10 or the mobile application of the personal device 11 is started from displaying a dialog client interface 1 on the screen 16 upon receipt of the client's incoming request 2 by verbal inputting of the same by means of the microphone 18 and determining the parameters of the client's incoming request 3 by analyzing the keywords of the request using the recorded programs of algorithms of the virtual assistant. If an Internet connection is present and after the parameters of the client's incoming request are determined, the data from the uploaded databases are received 4, the databases exchange the data with external databases on-line, wherein the external databases represent external databases of pharmacy networks 21, external databases of electronic prescriptions of medical institutions 22, external pharmacology knowledge databases 23, external payment systems Visa/MasterCard 24 for making payments in the automated pharmacy stores and external payment systems APPLE PAY and/or GOOGLE PAY 25 for making payments in the mobile application. If there is no Internet connection, the data will be received 4 from the uploaded databases of the uploaded databases module 19 off-line. Upon receipt of the data 4 from the uploaded databases module 19, upon their exchange with the external databases 21, 22, 23, 24, 25 on-line or if there is no such exchange off-line, the client's personal data is processed 5 by reading and further processing the client's ID card or passport data, as well as the drug prescription prescribed by the physician, by means of the scanner 27, if the automated pharmacy store 10 is used, or by means of the camera 28, if the mobile application of the personal device 11 is used, followed by providing the client with the response to the request 6 in the form of the direct verbal response of the virtual assistant by means of the speaker 30, while at the same time displaying the configuration of the client's order on the screen 16. Next steps comprise selecting, by the client, an option for completing the order 7 displayed on the screen 16 by means of the microphone 18 verbally or by pushing the corresponding buttons on the screen 16, and paying for the order 8 by means of the external payment systems Visa/MasterCard 24 for making payments in the automated pharmacy stores or the external payment systems APPLE PAY and/or GOOGLE PAY 25 for making payments in the mobile application. Therewith, the client may make the payment in the automated pharmacy store both during placing the order in the automated pharmacy store and on the basis of the QR code when placing the pre-order without payment in the mobile application. After the order is paid in the automated pharmacy store or in the mobile application and the corresponding QR code is received, the pharmacy product and/or the analogue thereof is dispensed 9 by means of the dispenser 33 of the automated pharmacy store. If the dispenser 33 is not able to dispense the pharmacy product for technical reasons, the client will be able to receive his/her order in any other automated pharmacy store using the QR code 34 printed by the printer that is embedded in the pharmacy product dispensing module. After the pharmacy product is dispensed 9 or the corresponding QR code is printed, the operation of the automated pharmacy store is ended 35.

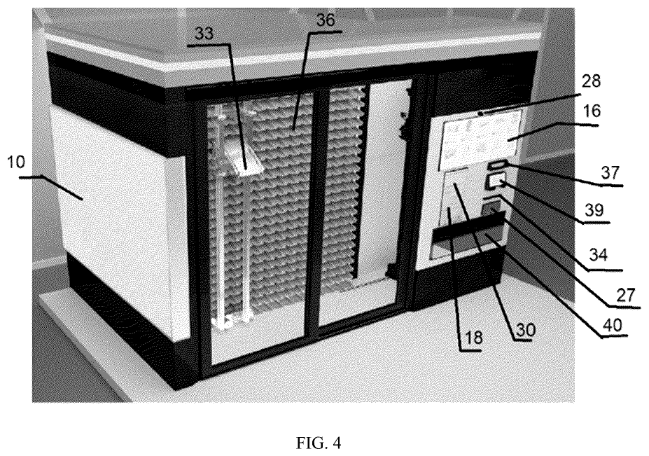

FIG. 4 shows a general view of the automated pharmacy store 10 for dispensing the pharmacy product and/or the analogue thereof, the automated pharmacy store comprises the touch screen 16, the microphone 18 for determining parameters of the client's incoming verbal request, the scanner 27 for scanning the prescription, ID card or passport, the video camera 28 for identifying the client's identity by comparing the image taken by the video camera to the photo in the passport or in the ID card, the speaker 30 for providing the client with the response to the request in the form of the direct verbal response of the virtual assistant, the dispenser 33, the printer for printing the QR code 34 that is also used to print receipts, a module 36 for storing the pharmacy products, a bill acceptor 37 for making cash payment, a bank card reader 39, a product dispensing window 40. It should be noted that the structure of the automated pharmacy store is merely illustrative. The automated pharmacy store is characterized by a modular structure that may comprise various number of modules 37 for storing the pharmacy products and their flexible arrangement, wherein one module may store at least 500 types of the pharmacy products and at least 6000 units to be loaded. Therewith, the automated pharmacy store having one module for storing the pharmacy products covers an area of about 2 m2, thereby enabling to locate such pharmacy store at any convenient place due to its compact dimensions.

Therefore, the claimed group of inventions enables to develop a method and a system reliable and 24/7 available due to a lack of a human-related factor influence, algorithmically simple and clear, the method and the system are for providing the client with the information regarding main characteristics of the pharmacy product and/or the analogue thereof in the automated pharmacy store or in the mobile application followed by dispensing the pharmacy product and/or the analogue thereof in the automated pharmacy store, which provide a maximum quick, accurate and reliable processing of the client's incoming request, and, thus, a timely provision of a maximum correct information regarding the main characteristics of the pharmacy product and/or the analogue thereof by means of the virtual assistant having pre-recorded programs of algorithms and fully uploaded pharmacology databases which are capable of being updated on-line, that does not require additional settings from the client and may operate in an autonomous mode, as well as characterized by a high level trust therefor by the client due to its maximum visual similarity of the virtual assistant to the real human, that is capable of providing the information to the client both verbally and visually at the same time.

It should be noted that the above-described embodiments of the group of inventions should be used as an illustration only and shall not limit the scope thereof. Obvious modifications of embodiments of the group of inventions may be easily made by persons skilled in this field of art without going beyond the essence thereof.

1. A method for providing a client with information regarding main characteristics of at least one product followed by dispensing thereof in an automated pharmacy store using at least one hardware processor and a memory for storing computer-executable instructions or a mobile application, the at least one product comprising at least one of a pharmacy product and an analogue thereof, the method comprising:
causing, by the client, a display on a screen of a dialog client interface via a graphical user interface (GUI) module simultaneously with a visual three-dimensional image of a virtual assistant having recorded programs of algorithms and uploaded databases that are capable of being updated on-line, wherein the update rate of the three-dimensional image of the virtual assistant is at least 60 Hz/sec;
receiving an incoming request from the client by a verbal input thereof;
determining parameters of the client's incoming request by means of a direct speech recognition module;
receiving data from the uploaded databases that are configured to exchange data with external databases at a data transfer rate of at least 4 MBit/sec;
processing a client's personal data by means of a personal data processing module;
providing a direct verbal response by the virtual assistant to the client in reply to the incoming request, the direct verbal response being provided using a speaker in operable communication with the GUI;
displaying, simultaneously with the direct verbal response, a configuration of a client's order on the screen of the dialog client interface of the GUI module;
selecting, by the client, an option for completing the order displayed on the screen of the dialog client interface of the GUI module;
paying, by the client, for the selected order using a payment processing module, the payment processing module comprising a magnetic card reader and a cash payment bill acceptor; and
physically dispensing, by a physical pharmacy product dispenser, the at least one product in the automated pharmacy store.

2. The method according to claim 1, wherein the at least one product comprises at least one of a pharmaceutical drug, an analogue of a pharmaceutical drug, a personal hygienic item, a medical item, a disinfectant, and a cosmetic agent.

3. The method according to claim 1, wherein the external databases represent databases of pharmacy networks and/or databases of electronic prescriptions of medical institutions and/or pharmacological knowledge databases and/or payment systems VISA and/or MasterCard and/or APPLE PAY and/or GOOGLE PAY.

4. The method according to claim 1, wherein the direct verbal response of the three-dimensional virtual assistant is provided by a computer program that is responsible for compliance with an order of actions of the virtual assistant in real-time.

5. The method according to claim 1, wherein the client's selection of the order displayed on the screen of the dialog client interface of the GUI module is a voice selection and/or a selection made by pushing buttons on a keyboard and/or a selection made by means of a touch screen.

6. A system for providing a client with information regarding main characteristics of at least one product followed by dispensing thereof in an automated pharmacy store using at least one hardware processor and a memory for storing computer-executable instructions or a mobile application, the at least one product comprising at least one of a pharmacy product and an analogue thereof, the system comprises:
a graphical user interface (GUI) module for providing on a screen a visual three-dimensional image of a virtual assistant having recorded programs of algorithms and uploaded databases that are capable of being updated on-line, wherein the update rate of the three-dimensional image of the virtual assistant is at least 60 Hz/sec;
a client's direct speech recognition module for determining parameters of a client's incoming verbal request;
a module that is configured to upload databases and to exchange data with external databases at a data transfer rate of at least 4 MBit/sec;
a client's personal data processing module;
a speaker in operable communication with the GUI;
a module for providing the client with a response to an incoming request;
a payment processing module, the payment processing module comprising a magnetic card reader and a cash payment bill acceptor; and
a physical pharmacy product dispenser configured to physically dispense the at least one product,
wherein the GUI is configured such that a direct verbal response is provided by the virtual assistant to the client using the speaker in reply to the client's incoming verbal request, and
wherein the GUI is further configured to, simultaneously with the direct verbal response, provide on the screen a configuration of a client's order.

7. The system according to claim 6, wherein the at least one product comprises a pharmaceutical drug, an analogue of a pharmacy drug, a personal hygienic item, a medical item, a disinfectant, and a cosmetic agent.

8. The system according to claim 6, wherein the external databases represent databases of pharmacy networks and/or databases of electronic prescriptions of medical institutions and/or pharmacological knowledge databases and/or payment systems VISA and/or MasterCard and/or APPLE PAY and/or GOOGLE PAY.
